(19) United States
(12) Patent Application Publication
(10) Pub. No.: US 2004/0076544 A1
(43) Pub. Date: Apr. 22, 2004

(54) Method and apparatus for scanning and sterilizing mail received at a drop box

(76) Inventor: Hung Dao, Arlington, TX (US)

Correspondence Address: Philip G. Meyers, Philip G. Meyers Law Office, Suite 302, 1009 Long Prairie Road, Flower Mound, TX, 75022, US

(21) Appl. No.: 10/682,002

(22) Filed: Oct. 9, 2003

Related U.S. Application Data
(60) Provisional application No. 60/417,116, filed on Oct. 9, 2002.

(51) Int. Cl. G01N 33/00

(52) U.S. Cl. 422/62

(57) ABSTRACT

A drop box for receiving mail pieces for collection and delivery by a postal service includes an enclosure having a slot sized for receiving mail pieces, a feeder positioned inside the drop box for engaging and conveying mail pieces inserted into the slot, a detector for detecting a potential contaminant inside the drop box, and a controller for initiating a preprogrammed action when a potential contaminant is detected inside the drop box. The preprogramed action may include locking the box and sounding an alarm coupled to the controller. The drop box may also be equipped with means for sterilizing the drop box and its contents such as a source of electromagnetic radiation and/or a source of a chemical sterilizing agent.

This application claims priority of U.S. Provisional Patent Application Serial No. 60/417,116, filed Oct. 9, 2002.

### TECHNICAL FIELD

The present invention relates to the field of mail handling and in particular to a method and apparatus for scanning and/or sterilizing mail pieces received at a drop box to prevent potentially hazardous material from entering a postal system.

### BACKGROUND OF THE INVENTION

Recently the United States Postal Service has recognized the need for techniques to sterilize the mail to prevent the mail from being used to perpetrate acts of bio-terrorism by sending germ-laden mail pieces through the mail. The USPS has recently taken steps at great expense to divert mail destined for certain U.S. government agencies for decontamination. However, before the mail can be diverted and sterilized, it must enter the postal system. Systems for diverting and/or sterilizing mail pieces do not address the hazards presented prior to the mail being received, diverted and sterilized. This is especially true in the case of unattended mail drop boxes and similar receptacles used by private couriers and delivery services. The term “drop box” as used herein generally refers to a receptacle wherein postal patrons and customers of private delivery services deposit mail and mail-like items for subsequent delivery. Such drop boxes are typically fixed in place, secured and configured such that mail or mail like items can be retrieved after deposit only through a locked door opened by a postal employee or employee of a delivery service having a key or similar means to unlock the drop box.

While the greatest potential risk is to the personnel that unload, transport and otherwise handle mail deposited in drop boxes, the public may also be exposed to potentially hazardous materials at drop boxes and Post Offices either by direct exposure to contaminated mail or indirectly through exposure to airborne contaminates and/or contact with contaminated surfaces.

There exists a need for scanning and/or sterilizing mail pieces entering postal systems that is focused on preventing mail pieces that may contain hazardous materials and in particular germ or spore laden materials from entering the system via unattended drop boxes and similar receptacles that are commonly used postal services and similar entities to receive mail pieces such as letters and thin packages for delivery.

### SUMMARY OF THE INVENTION

According to the invention, a drop box for receiving mail pieces for collection and delivery by a postal service includes an enclosure having a slot sized for receiving mail pieces, a door for closing the slot and a lock for securing the door. The drop box is equipped with a feeder positioned inside the drop box for engaging and conveying mail pieces inserted into the slot, a detection device disposed for measuring a physical property of a associated with potential contamination of the mail piece, and a control system operable to take an action when a potentially contaminated mail piece is detected by the detection device. In this regard, the drop box is equipped with means for sensing a mail piece inserted therein and activating the feeder in response. In other variations the drop box is provided with means for reversing the feeder when a potentially contaminated mail piece is detected and/or an alarm activated by the control system upon detection of a potentially contaminated mail piece. The drop box is also preferably provided with means for activating the lock when a potentially contaminated mail piece is detected.

In one aspect, the detection device is a scanner for forming an image of the mail piece and the control system includes a computer programmed with analysis logic to identify patterns associated with potentially contaminated mail pieces. Alternatively, the detection device measures a physical property of the mail piece and the control system includes pre-programmed logic to initiate an action when the measured physical property indicates a potentially contaminated mail piece. In yet another aspect, the drop box is provided with means for capturing and recording an image of individuals depositing mail pieces in the drop box.

In another variation a drop box for receiving mail pieces for collection and delivery by a postal service includes an enclosure having a slot sized for receiving mail pieces, a feeder positioned inside the drop box for engaging and conveying mail pieces inserted into the slot, and means for sterilizing mail pieces conveyed by the feeder. In this regard, the means for sterilization may be a source of electromagnetic radiation such as ebeam or X-ray frequency radiation or laser, maser or UV frequency radiation.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a front schematic view of a mail drop box according to the invention;

FIG. 2 is a cross section schematic view of the mail drop box of FIG. 1;

FIG. 3 is front schematic view of a second drop box in according to the invention; and

FIG. 4 is a cross section schematic view of the mail drop box of FIG. 3.

### DETAILED DESCRIPTION

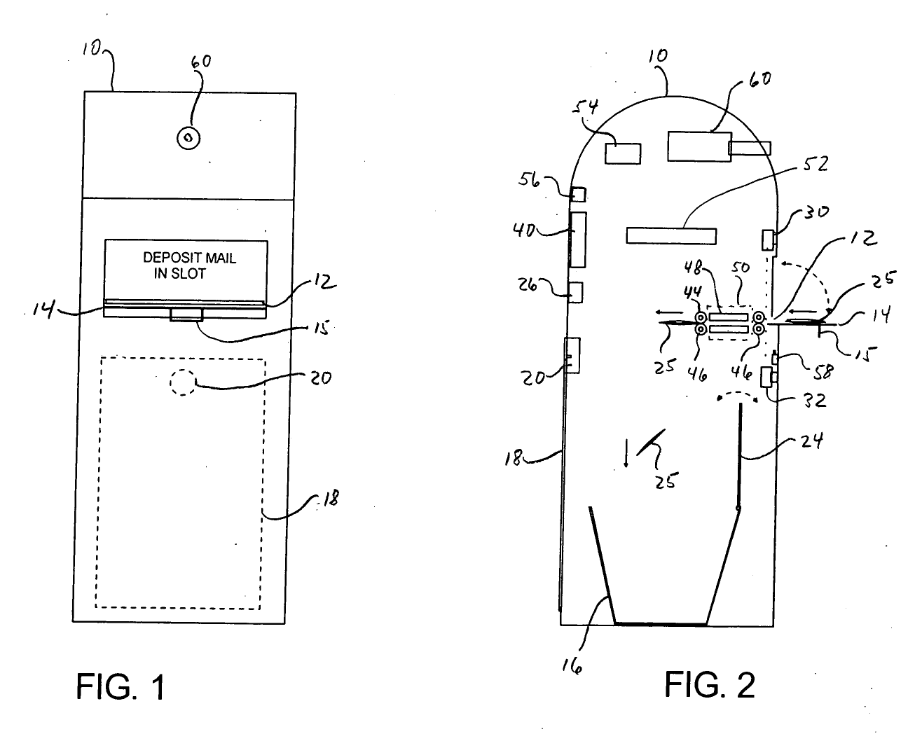

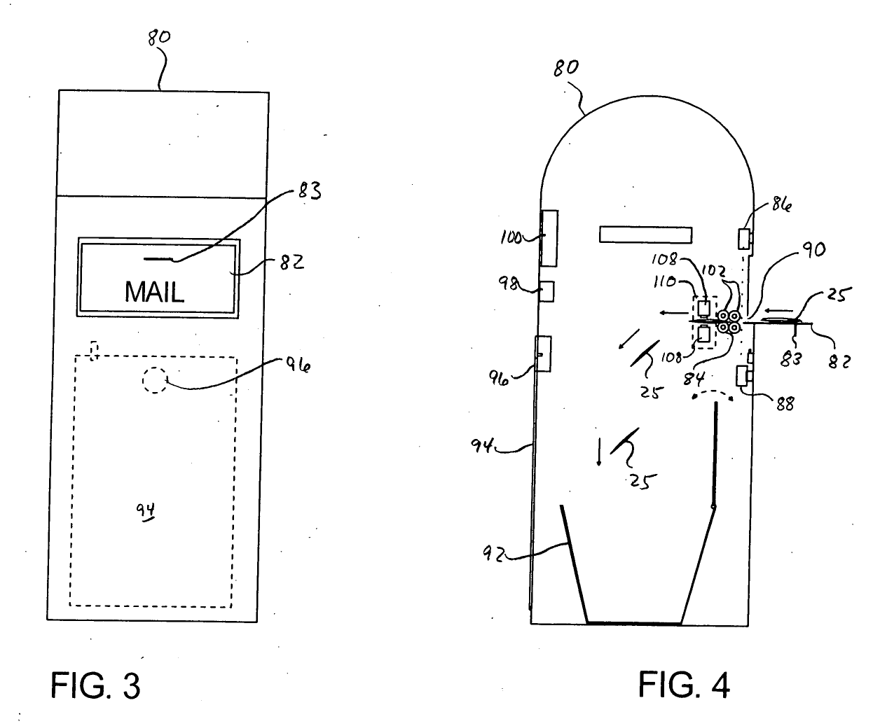

Referring to FIGS. 1 and 2, a mail piece drop box or receptacle 10 includes a mail deposit slot 12 where postal patrons may deposit mail pieces for collection by a mail service. Preferably, slot 12 is sized such that only relatively thin objects, such as mail pieces less than for example, one-fourth inch thick, may be inserted into the receptacle. Sizing slot 12 in this manner prevents individuals from inserting a finger or hand, oversized mail pieces, foreign objects and the like into drop box 10. Drop box 10 is provided with a pivoting door 14 with handle 15. Door 14 is held in a normal closed position (FIG. 3) by means of a spring or counterweight (not shown). As illustrated, door 14 in the open position is aligned with slot 12 to provide a platform upon which patrons may slide mail pieces through slot 12 into drop box 10.

A receiving container 16 is positioned in drop box 10 for receiving mail pieces 25 deposited by patrons through slot 12. A door 18 provides access to container 16 allowing mail service employees to remove container 16 and/or mail pieces 25 deposited by patrons. Door 18 is provided with a lock 20 to secure the receptacle and is preferably provided with a seal or gasket to prevent air borne contaminants from escaping from receptacle 10. Preferably, container 16 is provided with a sealable lid 24, so that the container may be sealed as or before it is removed from drop box 10 to prevent the escape of air born contaminants. Drop box 26 is also provided with an electrical power source 26 for powering the various components discussed below.

To detect when a mail piece is inserted through slot 12, drop box 10 is provided with a light source 30 which emits a beam of light to photo cell 32. When a mail piece is inserted through slot 12, the beam is interrupted and photo cell 32 transmits a signal to a controller 40 which activates feeder 44. As illustrated, feeder 44 comprises two pair of opposed rollers 46, at least one of which is powered. Feeder 44 engages mail pieces 25 and carries the mail pieces adjacent or through a sensor 50 for detection of a property or parameter associated with potentially dangerous contaminates as such as pathenogenic microorganisms, spores, toxins and similar hazardous materials. Although as described, light source 30 and photo cell 32 are used to sense a mail piece inserted into drop box 10, other sensing means such as a micro switch or proximity sensor may be utilized to perform this function.

In the illustrated embodiment, sensor 50 comprises an device such as an X-ray apparatus 48 that scans mail piece 25 to determine the density of the mail piece. Since many potentially dangerous contaminants are dust like materials that would tend to accumulate in the corners or along the edges of an envelope, variations in density along the width and length of the mail piece could be used to detect the possible presence of such materials. In this regard, it should be noted that there is currently no means of directly detecting the presence of potentially dangerous micro-organisms in an environment such as a drop box. Consequently, it is necessary to utilize technologies that detect a physical property or characteristic associated with such organisms and materials and/or devices that detect or measure the physical, electro-magnetic or electrochemical properties of a mail piece inserted into a drop box or the atmosphere present inside the drop box.

Referring again to FIGS. 1 and 2, when a suspicious variation in density of a mail piece is detected or observed, sensor 50 signals controller 40 which initiates an appropriate action. It will be appreciated that depending upon the complexity of the system, controller 52 may comprise one or more relays activated by sensor 50. Alternatively, controller 40 may comprise a microprocessor including preprogrammed instructions for responding to different input signals. For example, in the case where sensor 50 comprises an x-ray apparatus, an image of the mail piece based upon the density of the mail piece is controller 40, which in this case comprises a computer, that analyzes the image for variations or patterns that would indicate possible contamination. If the analysis indicates that the mail piece may be contaminated, controller 40 initiates one or more preprogramed responses, for example reversing feeder 44 to eject the mail piece 25 back through slot 12.

Controller 40 may also activate an alarm 54 when a potentially contaminated mail piece is detected. Alarm 54 may be an audible alarm such as a horn or siren and/or a flashing light. Controller 40 may also be provided with a communications link 56, such as a radio transmitter or telephone line for transmitting the alarm to alert the appropriate authorities. Additionally, controller 40 may also activate a emergency lock 58, locking door 14 to prevent air borne contaminates from escaping drop box 10.

It may be desirable to provide a means for identifying individuals depositing mail pieces in receptacle 10. Thus, as illustrated, receptacle 10 is provided with a camera 60 for capturing an image of patrons depositing mail in the receptacle. In one variation, camera 60 is a video camera connected to a video recorder that is operated continuously. Alternatively, camera 60 may connected to and actuated by photocell 32 and/or controller 40.

As an alternative to an X-ray scanner, sensor 50 may comprise another device capable of measuring one or more physical properties of the mail piece and its contents including magnetic, electric, electomagnetic, sonic, optical or dielectric properties of the mail piece to detect potentially hazardous material. For example, sensor 50 could measure the interaction of a magnetic field or electromagnetic wave with the of the mail piece and its contents. In this regard, the measurement of complex permittivity is discussed in U.S. Pat. No. 5,233,306, the disclosure of which is incorporated herein by reference for all purposes.

In another aspect, sensor 50 is a device capable of detecting the presence of various type of hazardous materials by sampling the air in drop box 10. In one variation, sensor 50 comprises a test filter sized to remove particles having a size greater than that of the microorganisms and/or carrier particles used to disperse the microorganisms. After filtering, the air sample is then passed through an optical detector to determine if particles pre-identified as potentially dangerous are present in the air. Methods used to identify such particles may vary as described further below, but in general it is desirable to characterize the target cells or particles as specifically as possible to avoid a high rate of false positive readings. In the embodiment wherein sensor 50 samples the air in drop box 10, drop box 10 may also be equipped with means for sterilizing the contents and atmosphere inside the box. Such means may comprise a dispenser 52 for a disinfectant agent or gas effective to destroy biological contaminants present in drop box 10.

Optical measurements include particle reflectance. U.S. Pat. No. 5,471,299 above describes an imaging system capable of recording both the radial and azimuthal (about the illuminating beam axis) variations in the pattern of scattered light from individual particles carried in a sample airflow. Particles are classified on the basis of their shape (whether, for example, spherical, cuboidal, flake-like, or fibrous) as well as on their size, the latter being derived from an assessment of the total scattered intensity. Thus loose powder used as a carrier for anthrax spores could be identified.

Sensor 50 may also use spectroscopic analysis to detect absorption spectra characteristic of known target pathogens such as anthrax. See, for example, U.S. Pat. No. 5,512,490 which describes an optic sensing apparatus and methodology for detecting and evaluating one or more analytes or ligands of interest, either alone or in admixture. The optic sensor of the system is comprised of a supporting member and an array formed of heterogeneous, semi-selective thin films which function as sensing receptor units and are able to detect a variety of different analytes and ligands using spectral recognition patterns. The entire contents of U.S. Pat. Nos. 5,512,490, 5,471,299, 5,320,814, 5,409,666, 5,382,512, 4,818,103, 5,093,866, and 4,606,636, describing systems for identifying unknown particles, are incorporated herein by reference for all purposes. PCT publication WO 00/63673 discloses a further system for identifying the size, shape and fluorescence of fluidborne particles.

If it is desired to identify specific microorganisms that pose a threat, such as bacteria, spores and viruses, sensor 50 may incorporate an bioassay system therein, generally wherein a reaction can occur between a pair of specifically binding substances such as an antigen and an antibody. One substance of the pair is presented on a solid phase such as a polymeric microbead. If the target substance is present in the sample, a specific binding reaction will occur and the reaction is then detected by any of a variety of known means. Almost all such systems operate in an aqueous phase, and as such it may be necessary to pass the potentially contaminated air from plenum 38 through an exchanger wherein airborne particles enter into the aqueous phase and may be tested for, optionally with further processing to break whole cells down into components substances.

Bioassay systems are well known and effective, but are usually run in a lab environment. Recent efforts have been directed towards making such systems more useful for detecting multiple target substances at a time with an electronic endpoint, that is, a change in properties of the system that can be measured electronically without resort to means such as chemical labels to detect that a reaction has occurred. Accordingly, sensor 30 may comprise an electronic “nose,” “tongue” or similar device adapted to sense the presence of particular microorganisms either directly or indirectly through detection of a substance associated with the potentially dangerous microorganisms, such as a carrier powder or a byproduct produced by the microorganism. These devices generally comprise a substance that evidences a electronically measurable change upon reacting with the microorganism or a material associated with the microorganism. The substance may change color, conductivity or fluoresce upon reacting with a suspicious substance. A number of sensor systems for detection of such substances in a fluid have been proposed, including those described in PCT Publication Nos. WO0155704, WO0155703, WO0155702, WO0155701, WO0106253, WO0106244, WO0106239, WO0068670, and WO0004372.

For example, PCT Publication Nos. WO0106239 provides a system for detecting analytes that includes a light source, a sensor array, and a detector. The sensor array is formed of a supporting member which is configured to hold a variety of chemically sensitive particles in an ordered array. The particles create a detectable signal in the presence of an analyte. The particles may produce optical (e. g., absorbance or reflectance) or fluorescence/phosphorescent signals upon exposure to an analyte. Examples of such particles include functionalized polymeric beads, agarous beads, dextrose beads, polyacrylamide beads, control pore glass beads, metal oxides particles (e. g., silicon dioxide (SiO₂) or aluminum oxides (Al₂O₃)), polymer thin films, metal quantum particles (e. g., silver, gold, platinum, etc.), and semiconductor quantum particles (e. g., Si, Ge, GaAs, etc.). A detector (e. g., a charge-coupled device“CCD”) is positioned below the sensor array to allow for data acquisition. Light originating from the light source may pass through the sensor array and out through the bottom side of the sensor array. Light modulated by the particles may pass through the sensor array and onto the proximally spaced detector. Evaluation of the optical changes may be completed by visual inspection or by use of a CCD detector by itself or in combination with an optical microscope. A microprocessor may be coupled to the CCD detector or the microscope.

A fluid delivery system may be coupled to the supporting member of the sensor array to introduce samples into and out of the sensor array. In one configuration, the sensor array system includes an array of particles. The particles may include a receptor molecule coupled to a polymeric bead. The receptors are chosen for interacting with analytes. This interaction may take the form of a binding/association of the receptors with the analytes. The supporting member may be made of any material capable of supporting the particles, while allowing the passage of the appropriate wavelengths of light. The supporting member may include a plurality of cavities. The cavities may be formed such that at least one particle is substantially contained within the cavity.

A high sensitivity CCD array is used to measure changes in optical characteristics which occur upon binding of the biological/chemical agents. The CCD arrays may be interfaced with filters, light sources, fluid delivery and micromachined particle receptacles to create a functional sensor array. Data acquisition and handling is performed with existing CCD technology. CCD detectors may be configured to measure white light, ultraviolet light or fluorescence. Other detectors such as photomultiplier tubes, charge induction devices, photo diodes, photodiode arrays, and microchannel plates may also be used. A particle of this system possesses both the ability to bind the analyte of interest and to create a modulated signal. The particle has receptor molecules which posses the ability to bind the analyte of interest and to create a modulated signal. Alternatively, the particle may include receptor molecules and indicators.

Upon binding the analyte of interest, the receptor molecule causes the indicator molecule to produce the modulated signal. The receptor molecules may be naturally occurring or synthetic receptors. Some examples of natural receptors include, but are not limited to, DNA, RNA, proteins, enzymes, oligopeptides, antigens, and antibodies. Either natural or synthetic receptors may be chosen for their ability to bind to the analyte molecules in a specific manner. In one embodiment, a naturally occurring or synthetic receptor is bound to a polymeric bead in order to create the particle. The particle, in some embodiments, is capable of both binding the analyte (s) of interest and creating a detectable signal. In some instances, the particle creates an optical signal when bound to an analyte of interest.

A variety of natural and synthetic receptors may be used. The synthetic receptors may come from a variety of classes including, but not limited to, polynucleotides (e. g., aptamers), peptides (e. g., enzymes and antibodies), synthetic receptors, polymeric unnatural biopolymers (e. g., polythioureas, polyguanidiniums), and imprinted polymers. Polynucleotides are relatively small fragments of DNA which may be derived by sequentially building the DNA sequence. Peptides include natural peptides such as antibodies or enzymes or may be synthesized from amino acids. Unnatural biopolymers are chemical structure which are based on natural biopolymers, but which are built from unnatural linking units. For example, polythioureas and polyguanidiniums have a structure similar to peptides, but may be synthesized from diamines (i. e., compounds which include at least two amine functional groups) rather than amino acids. Synthetic receptors are designed organic or inorganic structures capable of binding various analytes.

In order to identify, sense, and quantitate the presence of various bacteria using the micromachined sensor, two strategies may be used. First, small molecule recognition and detection may be exploited. Since each bacteria possesses a unique and distinctive concentration of the various cellular molecules, such as DNA, proteins, metabolites, and sugars, the fingerprint (i.e., the concentration and types of DNA, proteins, metabolites, and sugars) of each organism is expected to be unique. Hence, the analytes obtained from whole bacteria or broken down bacteria may be used to determine the presence of specific bacteria. A series of receptors specific for DNA molecules, proteins, metabolites, and sugars may be incorporated into an array. A solution containing bacteria will provide a pattern within the array which may be unique for the individual bacteria. In this manner, the presence of bacteria within a fluid may be determined. Bacteria may be detected as whole entities. To detect, sense, and identify intact bacteria, the cell surface of one bacteria is differentiated from other bacteria. One method of accomplishing this differentiation is to target cell surface oligosaccharides (sugar residues). Each bacterial class (gram negative, gram positive, etc.) displays a different oligosaccharide on their cell surfaces. The oligosaccharide, which is the code that is read by other cells giving an identification of the cell, is part of the cell-cell recognition and communication process. The use of synthetic receptors which are specific for oligosaccharides may be used to determine the presence of specific bacteria by analyzing for the cell surface oligosaccharides.

A system such as the foregoing may be adapted for purposes of the invention to detect for dangerous bacteria, spores or the like. Since such a system is based on specific binding reactions between the target and the receptor, it is less likely to result in false positives than a system based on optical particle recognition, but may be more difficult to implement.

Turning now to FIGS. 3 and 4, in a second variation a mail drop box 80 includes a pivoting door 82 with handle 83, a mail feeder 84, a light source 86 and photo cell 88 for detecting a mail piece inserted through a slot 90, and a receiving container 92, all essentially as described in connection with drop box 10 of FIGS. 1 and 2. Drop box 80 also includes access door 94 and lock 96 for securing the door. An electrical source 98 provides power for feeder 84, light source 86, photo cell 88 and controller 100, along with any other components requiring power.

Referring to FIG. 4, a mail piece inserted in drop box 80 is detected with photo cell 88 which transmits a signal to controller 100 which activates feeder 84. As in the case of drop box 10, feeder 84 comprises rollers 102, at least one of which is powered to engage and convey mail piece 25 through a sterilization unit 110 for elimination of any potentially dangerous biological materials. Although in the illustrated embodiments, feeders 44 and 84 comprise roller conveyors, it is contemplated that other means of conveying thin flat articles, such as belt conveyors or air conveyors may be utilized in the practice of the invention.

In one variation, sterilization unit 110 comprises one or more sources 108 of electromagnetic radiation configured to deliver a dosage of radiation effective to destroy biological contaminants present on and in mail pieces 25. Sterilization unit 110 may generate electromagnetic radiation within a broad spectrum of frequencies including RF, microwave, UV, laser, maser, electron bean and X-ray. Electromagnetic radiation at frequencies and powers requiring large amounts of shielding are less preferred, but may be utilized under some circumstances. Sterilization unit 110 may utilize an electric, magnetic or combined electrical and magnetic field to destroy biological material on in mail pieces processed through the unit. For example, sterilization unit 110 may comprise one or more lasers or masers operating on a frequency known to destroy biological material, an RF or micro wave source, a UV source or similar device.

In another variation, sterilization unit 110 utilizes a gas, disinfectant mist, laser or similar means to sterilize the exterior surfaces of the mail piece. While sterilization of the exterior of the mail piece may not as desirable as sterilization of the mail piece and its contents, it may be desirable as a first treatment designed primarily to protect mail service personnel that collect the mail pieces prior to subsequent processing which may include more extensive treatment.

The features of drop box 10 may be combined with those of drop box 80. For example, drop box 80 may be equipped with a sensor for detecting a potentially contaminated mail so that sterilization unit 110 is utilized only when a contaminated mail piece is detected. Drop box 80 may also be equipped with a camera, similar or identical to camera 54, for capturing images of patrons depositing mail pieces in drop box 80. Drop box 80 may also be provided with a sensor for detecting the presence of biological materials in the atmosphere inside drop box 80 and signaling controller 100 when a potential contaminant is detected in the air inside the drop box. In this case, drop box 80 may also be equipped with an alarm and lock, similar or identical to alarm 54 and lock 58 of drop box 10 activated by controller 1, as well as a disinfectant dispenser 52.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is, therefore, intended that the appended claims encompass any such modifications or embodiments.

1. A drop box for receiving mail pieces for collection and delivery by a postal service comprising:
an enclosure having a slot sized for receiving mail pieces;
a feeder positioned inside the drop box for engaging and conveying mail pieces inserted into the slot;
a detection device disposed for measuring a physical property of a mail piece engaged by the feeder, which property is associated with potential contamination of the mail piece; and
a control system operable to take an action when a potentially contaminated mail piece is detected by the detection device.

2. The drop box of claim 1 further comprising means for reversing the feeder when a potentially contaminated mail piece is detected.

3. The drop box of claim 1 further comprising means for sensing a mail piece inserted therein and activating the feeder when a mail piece is inserted into the slot.

4. The drop box of claim 3 further comprising means for reversing the feeder when a potentially contaminated mail piece is detected.

5. The drop box of claim 1 further comprising alarm means activated by the control system upon detection of a potentially contaminated mail piece.

6. The drop box of claim 1 wherein the detection device is a scanner forming an image of the mail piece and the control system includes a computer programmed with analysis logic to identify patterns associated with potentially contaminated mail pieces.

7. The drop box of claim 1 wherein the detection device measures a physical property of the mail piece and the control system includes pre-programmed logic to initiate an action when the measured physical property indicates a potentially contaminated mail piece.

8. The drop box of claim 7 further comprising a door for closing the slot and a lock for securing the door and means for activating the lock and wherein the initiated action is activation of the lock.

9. The drop box of claim 7 further comprising alarm means and wherein the initiated action is activating the alarm means.

10. The drop box of claim 1 further comprising means for capturing and recording an image of individuals depositing mail pieces in the drop box.

11. A drop box for receiving mail pieces for collection and delivery by a postal service comprising:
an enclosure having a slot sized for receiving mail pieces;
a feeder positioned inside the drop box for engaging and conveying mail pieces inserted into the slot;
means for sterilizing mail pieces conveyed by the feeder.

12. The drop box of claim 11 further comprising sensor for activating the feeder when the sensor detects a mail piece inserted in the drop box.

13. The drop box of claim 11 wherein the means for sterilizing mail pieces comprises a source of electromagnetic radiation.

14. The drop box of claim 13 wherein the electromagnetic radiation is ebeam or X-ray frequency radiation.

15. The drop box of claim 13 wherein the electromagnetic radiation is laser, maser or UV frequency radiation.

16. The drop box of claim 11 further comprising means for detecting a potential contaminant inside the drop box.

17. The drop box of claim 16 further comprising a controller for initiating a preprogramed action when a potential contaminant is detected inside the drop box.

18. 11. A drop box for receiving mail pieces for collection and delivery by a postal service comprising:
an enclosure having a slot sized for receiving mail pieces;
a feeder positioned inside the drop box for engaging and conveying mail pieces inserted into the slot;
means for detecting a potential contaminant inside the drop box; and
a controller for initiating a preprogrammed action when a potential contaminant is detected inside the drop box.

19. The drop box of claim 18 further comprising an alarm and wherein the preprogrammed action is activating the alarm upon detection of a potential contaminant.

20. The drop box of claim 18 further comprising means for sterilizing the interior of the drop box and its contents and wherein the preprogrammed action is activating the sterilization means.
